(19) United States
(12) Patent Application Publication
(10) Pub. No.: US 2004/0143232 A1
(43) Pub. Date: Jul. 22, 2004

(54) Circumpak

(76) Inventors: Florantina Perez, Miami, FL (US); Seymour Shor, Ft. Lauderdale, FL (US)

Correspondence Address: Florantina Perez, 7994 Grand Canal Drive, Miami, FL, 33144, US

(21) Appl. No.: 10/208,712

(22) Filed: Jan. 21, 2003

(51) Int. Cl. A61F 13/15; A61F 13/20

(52) U.S. Cl. 604/385.09

(57) ABSTRACT

Circumpak is standard diaper approximately 13.5″×18″ L, which Has been altered. Circumpak has an oblong hole in the center, 1.5″ L×0.75″ W. This allows penis to be separated from anus and bowel movements. This has the ability to dramatically reduce infection after circumcision has Been performed. Cirmcumpak has been on trail basis in South Miami Dade Hospital for over 14 months. It has been proven to be very effective and it dramatically reduces cause of infection after circumsision.

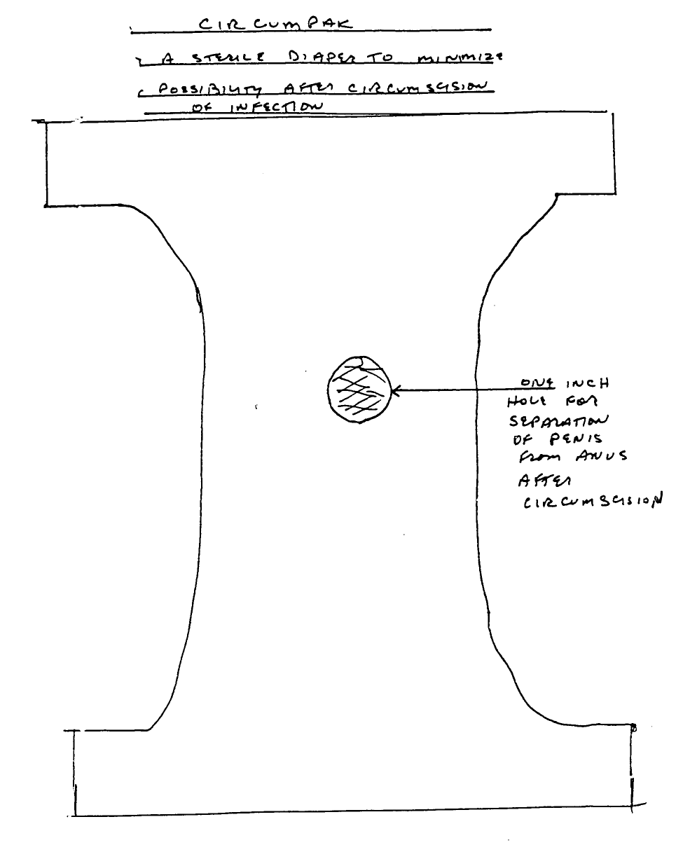

Over 85% of physicians are requesting the use of this diaper for their Patients when performing circumcision in this facility. It has the Endorsement of staff as well as administration of this hospital.

Circumpak is a diaper approximately 13.5″WX18.0″L it has been altered with a 1″ to 1.5″ hole, the hole maybe circular, oblong, or rectangular.

This allows for the separation of the feces from the circumcised penis to dramatically reduce infection. Secondary diaper may be changed after urination without disturbing the primary diaper itself.

As per your last letter, we claim:

1) A Circumpak diaper is a product that Dramatically reduces the possibility of infection after circumcision. It has been in use over twelve (12) Months at South Miami Dade Hospital.

2) Approximately eighty to ninety percent (80%-90%) of the physicians performing this procedure states that it dramatically reduces infection.
